(19) United States
(12) Patent Application Publication
(10) Pub. No.: US 2023/0130987 A1
(43) Pub. Date: Apr. 27, 2023

(54) SYSTEMS, METHODS, AND DEVICES FOR PRESERVING PATIENT PRIVACY

(71) Applicant: EMED LABS, LLC, Miami, FL (US)

(72) Inventors: Nicholas Atkinson Kramer, Wilton Manors, FL (US); Adam Charles Carlson, Miami, FL (US); Samantha Eve Rassner, Pompano Beach, FL (US); Ryan Adam Martin, Sunrise, FL (US); John Ray Permenter, Miami, FL (US); Sam Miller, Hollywood, FL (US); Zachary Carl Nienstedt, Wilton Manors, FL (US); James Thomas Heising, Richland, WA (US)

(21) Appl. No.: 18/049,407

(22) Filed: Oct. 25, 2022

Related U.S. Application Data
(60) Provisional application No. 63/263,075, filed on Oct. 26, 2021.
(60) Provisional application No. 63/268,672, filed on Feb. 28, 2022.
(60) Provisional application No. 63/367,429, filed on Jun. 30, 2022.
(60) Provisional application No. 63/273,058, filed on Oct. 28, 2021.
(60) Provisional application No. 63/266,220, filed on Dec. 30, 2021.

(51) Int. Cl. G05D 1/00; G05D 1/02

(52) U.S. Cl. CPC G05D 1/0088; G05D 1/0246

(57) ABSTRACT

This disclosure relates to systems, methods, and devices for patient privacy and healthcare productivity enhancement. In some embodiments, a method can include receiving a request to begin a remote medical session, initiating the remote medical session, receiving a plurality of feature vectors representative of one or more images, and generating one or more reconstructed images using the received feature vectors. In some embodiments, a patient can respond to one or more questions. In some embodiments, the patient's responses can be automatically evaluated.

### PRIORITY APPLICATIONS

This application claims the benefit of U.S. Provisional Patent Application No. 63/263,075, titled “SYSTEMS AND METHODS FOR PRESERVING PATIENT PRIVACY,” filed Oct. 26, 2021, U.S. Provisional Patent Application No. 63/268,672, titled “SYSTEMS AND METHODS FOR PRESERVING PATIENT PRIVACY,” filed Feb. 28, 2022, U.S. Provisional Patent Application No. 63/367,429, titled “SYSTEMS METHODS AND DEVICES FOR SEMANTIC RELEVANCE CURATION TOOLCHAIN FOR ASYNCHRONOUS MEDICAL PRACTITIONER EFFICIENCY,” filed Jun. 30, 2022, U.S. Provisional Patent Application No. 63/273,058, titled “VOUCHER SERVICES,” filed Oct. 28, 2021, and U.S. Provisional Patent Application No. 63/266,220, titled “VOUCHER SERVICES,” filed Dec. 30, 2021, the contents of each of which are incorporated by reference herein. Any and all applications for which a foreign or domestic priority claim is identified in the Application Data Sheet as filed with the present application are hereby incorporated by reference under 37 CFR 1.57.

### BACKGROUND

### Field

The present application is directed to remote testing sessions. Some embodiments are directed to protecting user privacy during a remotely administered diagnostic test. Some embodiments relate to semantic relevance curation toolchains for asynchronous medical practitioner efficiency. In some embodiments, systems, methods, and devices can be configured to automatically detect relevant portions of a video conferencing session to create a supercut of the session. Some embodiments relate to providing voucher services to clients.

### Description

Use of telehealth to deliver healthcare services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the capture of video of the user. In some cases, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring proper sample collection, ensuring test verification and integrity, providing test result reporting to appropriate authorities and medical providers, protecting user privacy, and connecting testing users with medical providers, who are sometimes needed to provide guidance and/or oversight of the testing procedures remotely.

### SUMMARY

While remote or at home health care testing offers many benefits, there are significant privacy risks associated with capturing and storing images, video, or other personal identifying information of users. For example, if video of testing sessions were to fall into the hands of an outside actor, user privacy could be compromised, and a company or organization offering remote medical testing could face significant financial, reputational, legal, and regulatory risks. Some users may prefer that even a testing company and/or proctor not have access to images, video, or other personal identifying information of the user.

Some embodiments describe systems, methods, and devices for preserving user privacy during remote testing sessions. These embodiments may reduce or eliminate the risk that identifying information could be vulnerable to discovery by unauthorized parties and/or may protect users of remote testing from exposure even to people associated with the testing company or organization. Additionally, some of the embodiments described herein may be used to reduce the amount of data that must be transmitted from a user's device.

In some embodiments, a system can conduct a telehealth video session with a patient for a doctor visit. The visit may be a follow-up visit relating to a treatment, or condition of the patient. The system can record a video of the patient answering one or more questions related to the treatment or condition. The system can use a semantic engine to automatically determine which portions of the patient's responses are relevant to the one or more questions. The system can automatically split up the video into a supercut. The supercut can be automatically sent to a doctor for viewing. The supercut can be shorter than the entire video of the patient, and the supercut can contain only the portions of the video relevant to the questions. Therefore, the doctor can spend less time with each patient while still having access to the information necessary for the patient's care. Doctors can see more patients in a certain period of time increasing the efficiency of each doctor. The system can provide patients with responses from the doctor which can contain recorded and/or prerecorded video segments combined in order to provide the patients with the instructions and care they need.

In some aspects, the techniques described herein relate to a method including: receiving, by a computing system, from a user device, a request to begin a remote medical session; initiating, by the computing system, the remote medical session; receiving, by the computing system, from the user device, a plurality of feature vectors representative of one or more images; and generating, by the computing system, one or more reconstructed images using the received feature vectors.

In some aspects, the techniques described herein relate to a method, further including: transmitting, by the computing to a proctor computing device, the one or more reconstructed images.

In some aspects, the techniques described herein relate to a method, further including: storing, by the computing system in a non-volatile memory, at least one of the plurality of feature vectors of the one or more reconstructed images.

In some aspects, the techniques described herein relate to a method, further including: generating, by the computing system based at least in part on the one or more reconstructing images, a video.

In some aspects, the techniques described herein relate to a method, wherein generating the video includes applying a physics engine to the plurality of feature vectors.

In some aspects, the techniques described herein relate to a method, wherein generating the video includes applying a skeletal muscular model to the plurality of feature vectors.

In some aspects, the techniques described herein relate to a method, wherein generating the video includes estimating one or more missing feature vectors.

In some aspects, the techniques described herein relate to a method, wherein the estimating is performed using at least one of a physics engine or a skeletal muscular model.

In some aspects, the techniques described herein relate to a method, wherein generating the one or more reconstructed images includes: detecting one or more objects to exclude from the one or more reconstructed images; and excluding the one or more objects from the reconstructed images.

In some aspects, the techniques described herein relate to a method, further including: receiving, by the computing system from the user device, audio of the remote medical session.

In some aspects, the techniques described herein relate to a method, further including: generating, from the received audio, a transcript.

In some aspects, the techniques described herein relate to a method, wherein the audio includes one or more user responses to one or more questions, further including: determining, by the computing system using a semantic engine, a beginning of a user response; determining, by the computing system using the semantic engine, an end of the user response.

In some aspects, the techniques described herein relate to a method, further including: determining, by the computing system using the semantic engine, a type of the user response.

In some aspects, the techniques described herein relate to a method, further including: providing, by the computing system to the user, a question; receiving, by the computing system from the user, a response to the question; evaluating, by the computing system, the received response; and providing, by the computing system based at least in part on the evaluation, a response to the user.

In some aspects, the techniques described herein relate to a method, further including: determining, by the computing system based at least in part on the user response, a second question; and providing, by the computing system to the user, the second question.

In some aspects, the techniques described herein relate to a method, further including: determining, by the computing system, that there are no more questions to ask the user.

In some aspects, the techniques described herein relate to a method, further including: generating, by the computing system, a transcript of the user responses.

In some aspects, the techniques described herein relate to a method, further including: evaluating the user responses using a semantic engine.

In some aspects, the techniques described herein relate to a method, further including: generating, by the computing system, a supercut including at least part of one or more user responses.

In some aspects, the techniques described herein relate to a method, further including: receiving, by the computing system from the user device, one or more image frames; and training a machine learning model to extract feature vectors from the one or more image frames.

In some aspects, the techniques described herein relate to a system including: a non-transitory computer-readable medium with instructions encoded thereon; and one or more processors configured to execute the instructions to cause the system to perform steps including: receiving, from a user device, a request to begin a remote medical session; initiating the remote medical session; receiving, from the user device, a plurality of feature vectors representative of one or more images; and generating one or more reconstructed images using the received feature vectors.

In some aspects, the techniques described herein relate to a system, wherein the system is further configured to perform steps including: transmitting, to a proctor computing device, the one or more reconstructed images.

In some aspects, the techniques described herein relate to a system, wherein the system is further configured to perform steps including: storing, in a non-volatile memory, at least one of the plurality of feature vectors or the one or more reconstructed images.

In some aspects, the techniques described herein relate to a system, wherein the system is further configured to perform steps including: generating, based at least in part on the one or more reconstructing images, a video.

In some aspects, the techniques described herein relate to a system, wherein generating the video includes applying a physics engine to the plurality of feature vectors.

In some aspects, the techniques described herein relate to a system, wherein generating the video includes applying a skeletal muscular model to the plurality of feature vectors.

In some aspects, the techniques described herein relate to a system, wherein generating the video includes estimating one or more missing feature vectors.

In some aspects, the techniques described herein relate to a system, wherein the estimating is performed using at least one of a physics engine or a skeletal muscular model.

In some aspects, the techniques described herein relate to a system, wherein generating the one or more reconstructed images includes: detecting one or more objects to exclude from the one or more reconstructed images; and excluding the one or more objects from the reconstructed images.

In some aspects, the techniques described herein relate to a system, wherein the system is further configured to perform steps including: receiving, by the computing system from the user device, audio of the remote medical session.

In some aspects, the techniques described herein relate to a system, wherein the system is further configured to perform steps including: generating, from the received audio, a transcript.

In some aspects, the techniques described herein relate to a system, wherein the audio includes one or more user responses to one or more questions, further including: determining, using a semantic engine, a beginning of a user response; determining, using the semantic engine, an end of the user response.

In some aspects, the techniques described herein relate to a system, wherein the system is further configured to perform steps including: determining, using the semantic engine, a type of the user response.

In some aspects, the techniques described herein relate to a system, wherein the system is further configured to perform steps including: providing, to the user, a question; receiving, from the user, a response to the question; evaluating the received response; and providing, based at least in part on the evaluation, a response to the user.

In some aspects, the techniques described herein relate to a system, wherein the system is further configured to perform steps including: determining, based at least in part on the user response, a second question; and providing, by the computing system to the user, the second question.

In some aspects, the techniques described herein relate to a system, wherein the system is further configured to perform steps including: determining that there are no more questions to ask the user.

In some aspects, the techniques described herein relate to a system, wherein the system is further configured to perform steps including: generating a transcript of the user responses.

In some aspects, the techniques described herein relate to a system, wherein the system is further configured to perform steps including: evaluating the user responses using a semantic engine.

In some aspects, the techniques described herein relate to a system, wherein the system is further configured to perform steps including: generating a supercut including at least part of one or more user responses.

In some aspects, the techniques described herein relate to a system, wherein the system is further configured to perform steps including: receiving, from the user device, one or more image frames; and training a machine learning model to extract feature vectors from the one or more image frames.

In some aspects, the techniques described herein relate to a method including: receiving, by a computing system from a user, a request for a remote medical session; initiating, by the computing system, the remote medical session, wherein initiating the remote medical session includes capturing audio of the user; providing, by the computing system to the user, a question; receiving, by the computing system from the user, a response to the question; evaluating, by the computing system, the received response; and providing, by the computing system based at least in part on the evaluation, a response to the user.

In some aspects, the techniques described herein relate to a method, further including: determining, by the computing system based at least in part on the user response, a second question; and providing, by the computing system to the user, the second question; receiving, by the computing system from the user, a response to the second question; and evaluating, by the computing system, the response to the second question.

In some aspects, the techniques described herein relate to a method, further including: determining, by the computing system, that there are no more questions to ask the user.

In some aspects, the techniques described herein relate to a method, further including: generating, by the computing system, a transcript.

In some aspects, the techniques described herein relate to a method, further including: wherein evaluating the received response is performed using a semantic engine.

In some aspects, the techniques described herein relate to a method, further including: generating, by the computing system, a supercut including at least part of one or more user responses.

In some aspects, the techniques described herein relate to a method, wherein evaluating the received response includes computing a semantics Hamming distance signal of at least one word in the received response.

In some aspects, the techniques described herein relate to a method, wherein evaluating the received response includes determining a sentiment of the response.

In some aspects, the techniques described herein relate to a method, wherein evaluating the received response including matching a word of the response to a keyword.

In some aspects, the techniques described herein relate to a method, wherein evaluating the received response includes generating a signal representing a correlation between a first word or sentence and a second word or sentence.

In some aspects, the techniques described herein relate to a system including: a non-transitory computer-readable medium with instructions encoded thereon; and one or more processors configured to execute the instructions to cause the system to perform steps including: receiving, by a computing system from a user, a request for a remote medical session; initiating, by the computing system, the remote medical session, wherein initiating the remote medical session includes capturing audio of the user; providing, by the computing system to the user, a question; receiving, by the computing system from the user, a response to the question; evaluating, by the computing system, the received response; and providing, by the computing system based at least in part on the evaluation, a response to the user.

In some aspects, the techniques described herein relate to a system, wherein the system is further configured to perform steps including: determining, by the computing system based at least in part on the user response, a second question; and providing, by the computing system to the user, the second question; receiving, by the computing system from the user, a response to the second question; and evaluating, by the computing system, the response to the second question.

In some aspects, the techniques described herein relate to a system, wherein the system is further configured to perform steps including: determining, by the computing system, that there are no more questions to ask the user.

In some aspects, the techniques described herein relate to a system, wherein the system is further configured to perform steps including: generating, by the computing system, a transcript.

In some aspects, the techniques described herein relate to a system, wherein the system is further configured to perform steps including: wherein evaluating the received response is performed using a semantic engine.

In some aspects, the techniques described herein relate to a system, wherein the system is further configured to perform steps including: generating, by the computing system, a supercut including at least part of one or more user responses.

In some aspects, the techniques described herein relate to a system, wherein evaluating the received response includes computing a semantics Hamming distance signal of at least one word in the received response.

In some aspects, the techniques described herein relate to a system, wherein evaluating the received response includes determining a sentiment of the response.

In some aspects, the techniques described herein relate to a system, wherein evaluating the received response including matching a word of the response to a keyword.

In some aspects, the techniques described herein relate to a system, wherein evaluating the received response includes generating a signal representing a correlation between a first word or sentence and a second word or sentence.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one or more advantages taught herein without necessarily achieving other advantages as may be taught or suggested herein.

All of the embodiments described herein are intended to be within the scope of the invention herein disclosed. These and other embodiments will be readily apparent to those skilled in the art from the following detailed description, having reference to the attached figures. The invention is not intended to be limited to any particular disclosed embodiment or embodiments.

### BRIEF DESCRIPTION OF THE DRAWINGS

These and other features, aspects, and advantages of the present application are described with reference to drawings of certain embodiments, which are intended to illustrate, but not to limit, the present disclosure. It is to be understood that the attached drawings are for the purpose of illustrating concepts disclosed in the present application and may not be to scale.

FIG. 1 illustrates an embodiment of a system configured to enable a remote medical test.

FIGS. 2A-2C illustrate feature extraction and motion capture according to some embodiments.

FIG. 3 is a diagram that shows a testing session process according to some embodiments.

FIG. 4 is a diagram that shows a remote intake procedure according to some embodiments.

FIG. 5A is a block diagram illustrating an example voucher service protocol or method for a single client.

FIG. 5B is a block diagram illustrating an example voucher service protocol or method for multiple clients.

FIG. 6 illustrates an embodiment of a computer system that can be configured to perform one or more of the methods or processes described herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described. The term image as used herein is not intended to be limited to single still images, but may also include, for example, image frames from a video. As used herein, the terms “image” and “frame” are interchangeable.

As mentioned briefly above and as will now be explained in more detail and with reference to the drawings, some embodiments describe systems, methods, and devices for protecting user privacy during remote medical testing and/or for reducing data consumption during remote medical testing.

In some embodiments, it may be advantageous to reduce or eliminate the collection of personal identifiable information associated with remote medical testing. For example, storage of personal identifiable information presents significant financial, reputational, legal, and regulatory risks. It may be preferable, for example, to replace identifying information with another representation or abstraction of such information, such as replacing the user with an avatar in captured images and/or video. Conventional techniques to protect privacy such as background or facial blurring are imperfect and may at times lead to identifying information being revealed. For example, if a user's face is blurred, the user may move suddenly, causing a portion of the user's face to be shown momentarily. Moreover, such techniques may result in the loss of information that is important for monitoring remote medical testing.

In some embodiments, artificial intelligence (AI) and/or computer vision (CV) models may be used to extract features of the user such as gaze, facial expressions, eye location, head location, and other features, as well as data indicative of features in the user's environment such as furniture, walls, decorations, and the like. In some embodiments, it may be advantageous for a testing platform to receive video and perform feature extraction. For example, having access to the source video may make model training easier and/or faster. In other embodiments, it may be advantageous to conduct feature extraction on a user's device so that video of the user never needs to leave the user's device. For example, testing software on the user device may include a lightweight machine learning model that can perform feature extraction locally. Extracted features are devoid of personal identifiable information, and thus can be stored, used for model training, or for other purposes without the risk that user identities will be exposed.

In some embodiments, images and/or video may be reconstructed based on the extracted features. In some embodiments, proctors may review a reconstructed video instead of an original video that contains images of the user. In some embodiments, a proctor may view a combination of original video and reconstructed video. For example, a proctor may use original video to verify a user's identity or to observe one or more steps in the testing process that cannot be adequately captured using feature extraction techniques.

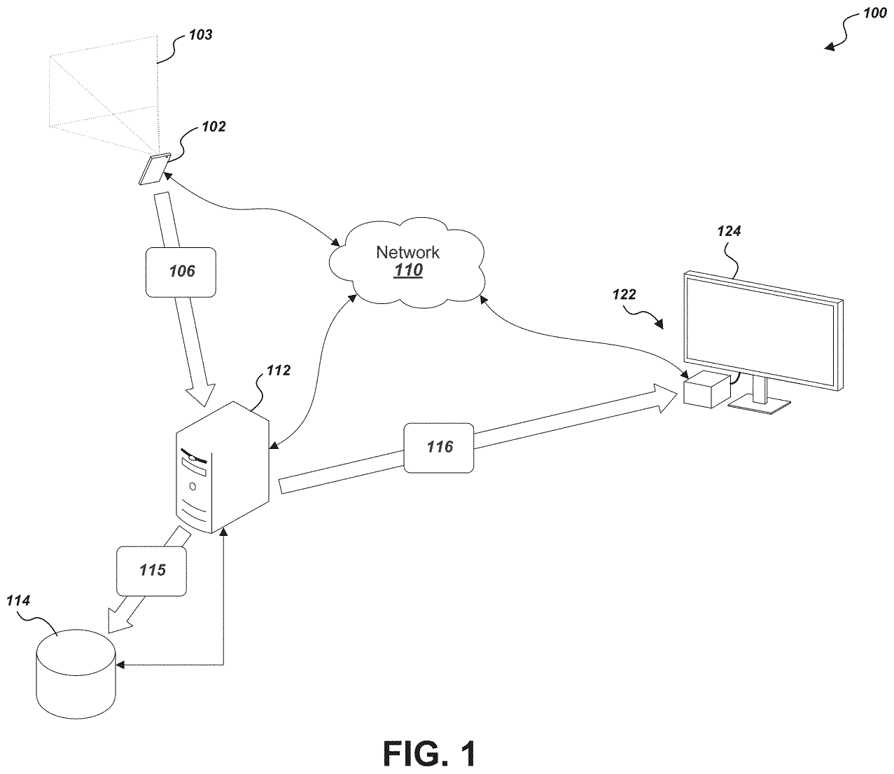

FIG. 1 illustrates an embodiment of a system 100 that could be used to take a remote medical test. The system 100 may include a user device 102 which may be associated with a user. The user device 102 may include a camera having a field of view (FOV) 103. The system may further comprise a testing platform 112 and, optionally, a database 114 that is communicatively coupled to the testing platform 112. The system 100 may further comprise a proctor computing device 122 with a display 124, and one or more networks 110 to which the user device 102, the testing platform 112, and the proctor computing device 122 are communicatively coupled. During operation, the user device 102 may capture images using its camera and may send data 106 to the testing platform 112 over the network 110. Based on receiving data 106 from the user device 102, the testing platform 112 may store data 115 into database 114 and transmit data 116 to the proctor computing device 122, which may operate display 124 based on receiving data 116 from the testing platform 112.

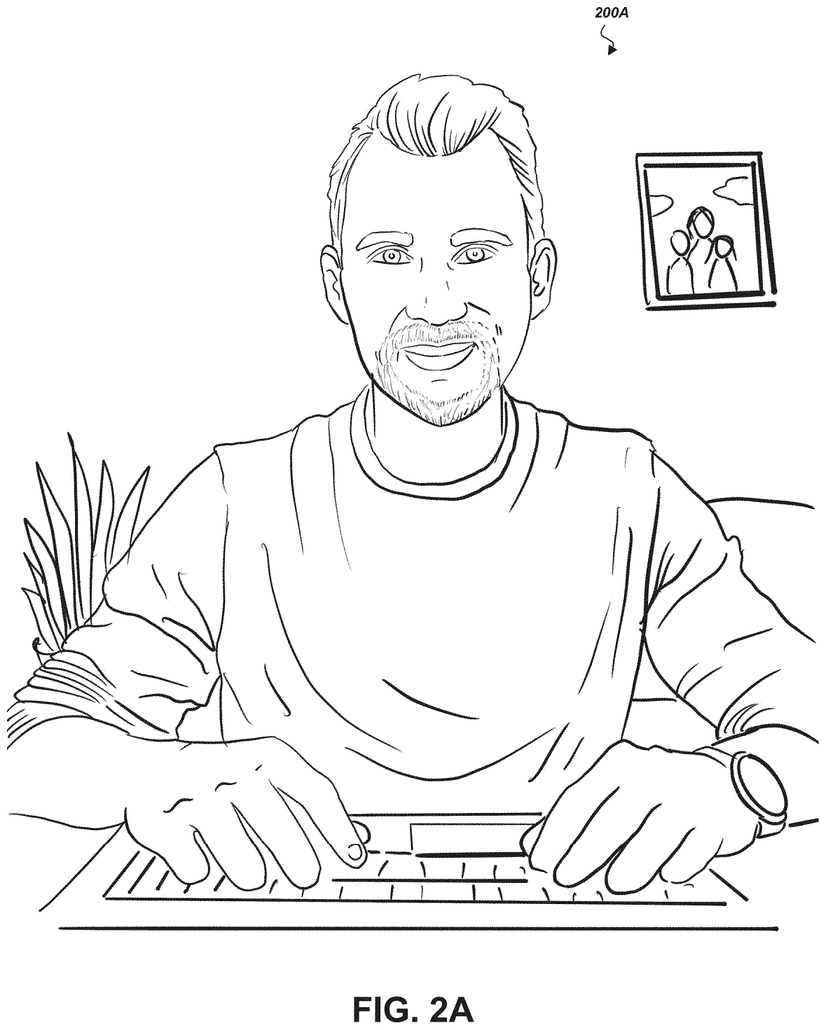

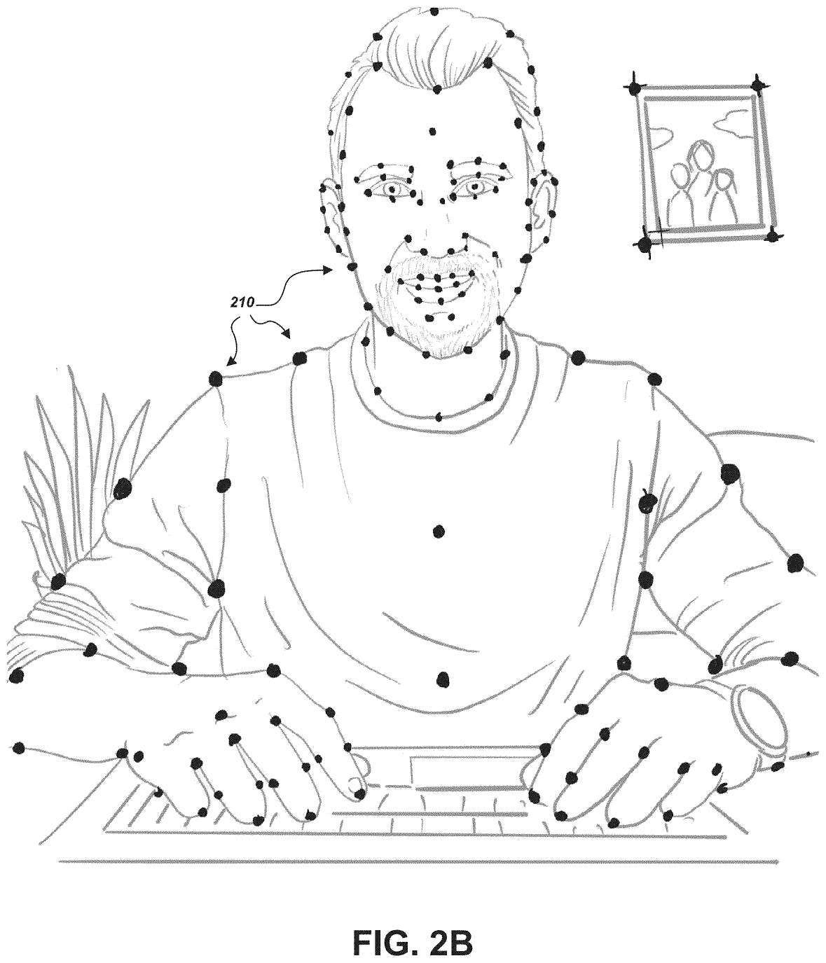

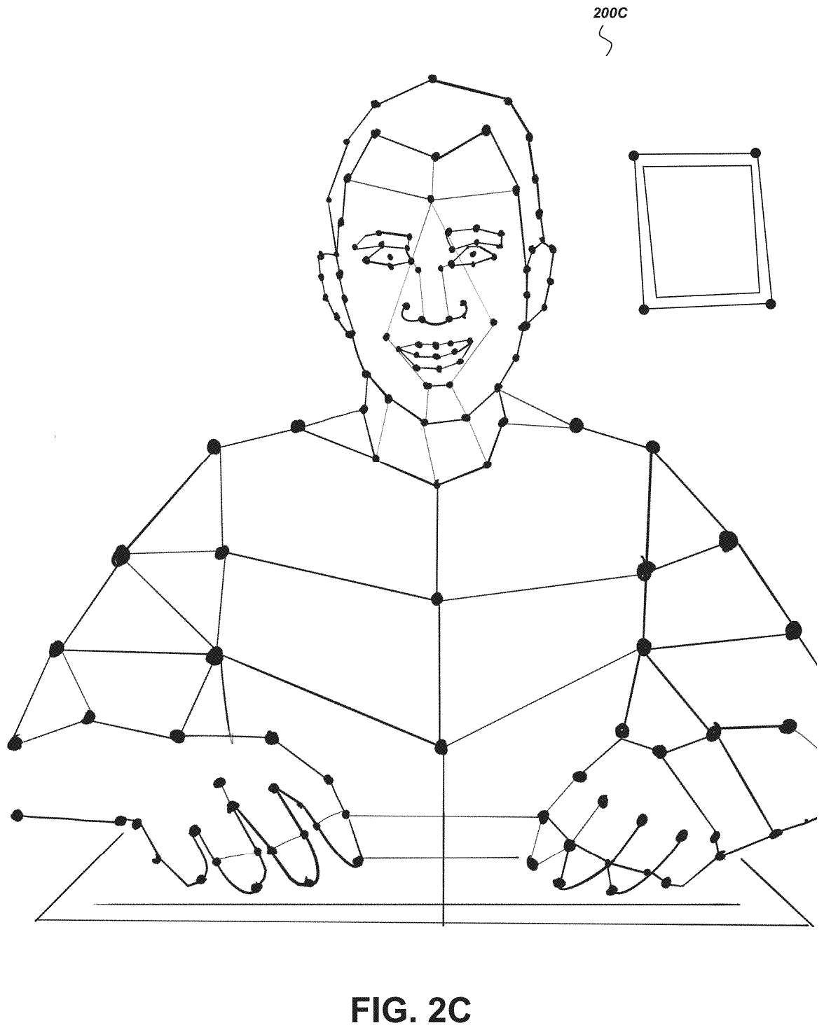

FIGS. 2A-2C illustrate feature extraction and motion capture according to some embodiments. FIG. 2A shows an example image 200A, which may be representative of an image captured using the camera of the user device 102. FIG. 2B shows an example set of feature vectors 210, which may be representative of features extracted from the example image 200A using, for example, a feature extraction model configured to receive images as input and, in response, extract feature vectors from the images and provide data representing sets of feature vectors as output. FIG. 2C shows an example reconstructed image 200C, which may be representative of an image reconstructed from the example set of feature vectors 210 using, for example, an image reconstruction model configured to receive data representing sets of feature vectors as input and, in response, generate one or more reconstructed images based on the sets of feature vectors.

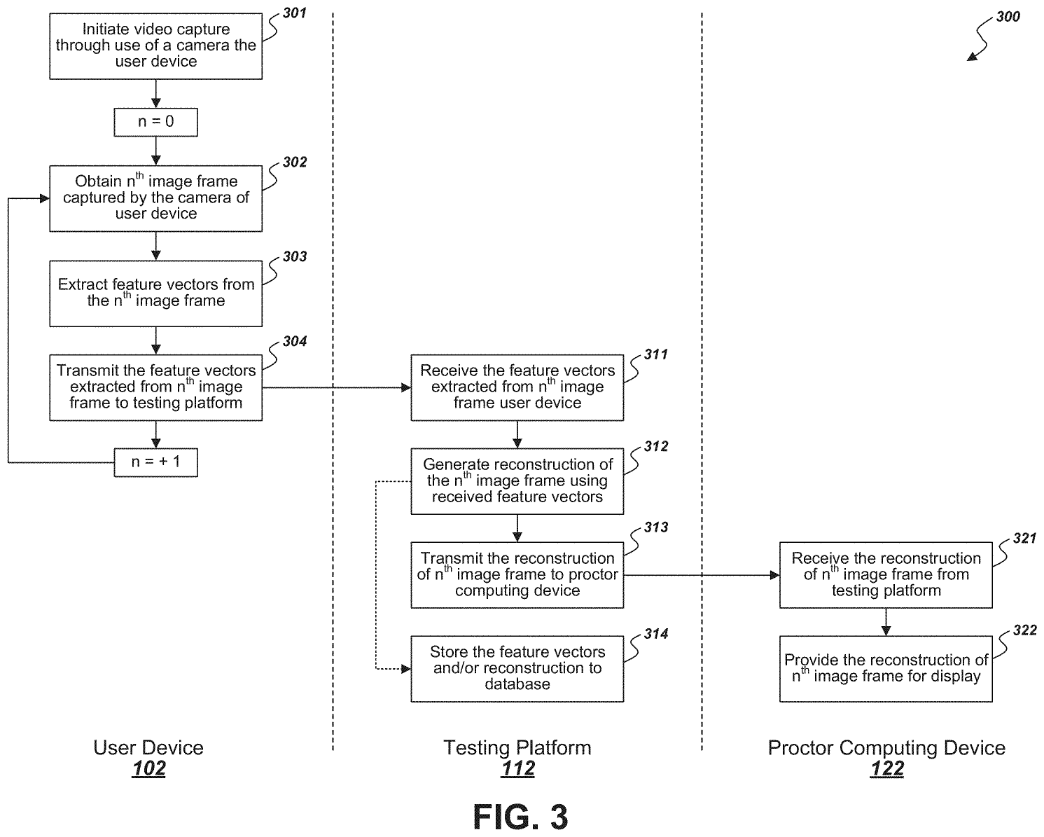

FIG. 3 is a diagram that shows a testing session process 300 according to some embodiments. The process 300 can begin, for example, when a patient requests to begin a medical session such as a testing session, follow up visit, or the like. At block 301, video capture through the camera of a user device 102 is initiated and a frame counter is initialized to zero. At block 302, an image frame is captured by the camera of the user device 102. At block 303, one or more feature vectors are extracted from the image frame, which are then transmitted at block 304 to the testing platform 112. The frame counter is then incremented and the process of capturing, identifying feature vectors, and transmitting the feature vectors to the testing platform 112 continues for each of one or more captured frames. At block 311, the testing platform 112 receives the feature vectors extracted from each of the one or more captured frames. At block 312, the testing platform generates reconstructions of each of the one or more captured frames using the received feature vectors. At block 313, each reconstructed frame is transmitted to the proctor computing device and, optionally, at block 314 the one or more feature vectors and/or reconstructed frames are stored in a database. At block 321, the proctor computing device 122 receives the one or more reconstructed frames from the testing platform 112. At block 322, the proctor computing device displays each of the one or more reconstructed frames. It will be understood that extracted features and/or reconstructed images do not have to be transmitted one at a time. For example, in some embodiments, a batch comprising a plurality of extracted features for a plurality of frames may be sent by the user device to the testing platform 112. In some embodiments, a batch of reconstructed images may be sent by the testing platform to the proctor computing device. The reconstructed images can be shown on the display of the proctor computing device. In some embodiments, the reconstructed images can be sent as individual images. In some embodiments, the reconstructed images can be sent as, for example, a video file or video stream. In some embodiments, the reconstructed images can be accompanied by audio.

FIG. 3 is an example embodiment, and other embodiments are possible. For example, in some embodiments a feature extraction model may reside at the testing platform and an image reconstruction model may reside at a computing device capable of communicating with a database. In some embodiments, a user device may transmit data representative of one or more images to the testing platform. In some embodiments, the testing platform may extract one or more feature vectors from the one or more images and transmit data representing the one or more feature vectors to the database. In some embodiments, the testing platform may transmit data representative of one or more images to a proctor computing device and the one or more images may be shown on the display of the proctor computing device.

In some embodiments, a feature extraction model may reside at the testing platform and an image reconstruction may reside at the testing platform. In some embodiments, the user device may transmit data representative of one or more images to the testing platform, which may then transmit data representing one or more sets of feature vectors and/or data representative of one or more reconstructed images to a database. In some embodiments, the testing platform may send data representative of one or more images to a proctor computing device. In some embodiments, the display of the proctor computing device may then show the one or more images.

In some embodiments, a feature extraction model may reside at the testing platform and an image reconstruction model may reside at the testing platform. In some embodiments, the user device may transmit data representative of one or more images to the testing platform, which may then transmit to the database data representing one or more sets of feature vectors and/or data representative of one or more reconstructed images. In some embodiments, the testing platform may send data representative of one or more reconstructed images to a proctor computing device, which may then be displayed on the display of the proctor computing device.

In some embodiments, a feature extraction model may reside on the testing platform. In some embodiments, an image reconstruction model may reside at the proctor computing device. In some embodiments, the user device may transmit data representative of one or more images to the testing platform. In some embodiments, the testing platform may transmit data representing one or more sets of feature vectors to a database. In some embodiments, the testing platform may transmit data representing one or more sets of feature vectors to a proctor computing device. In some embodiments, the image reconstruction model operating at the proctor computing device may generate one or more reconstructed images based on the one or more sets of feature vectors. The one or more reconstructed images may then be shown on the display device of the proctor computing device.

In some embodiments, a feature extraction model may reside on the user device. In some embodiments, an image reconstruction model may reside at a computing device capable of communicating with a database. In some embodiments, data representing one or more sets of feature vectors and data representative of one or more images may be transmitted by the user device to the testing platform. In some embodiments, the testing platform may transmit data representing one or more sets of feature vectors to a database. In some embodiments, the testing platform may transmit data representing one or more images to a proctor computing device. In some embodiments, the one or more images may be displayed on the display of the proctor computing device.

In some embodiments, a feature extraction model may reside on a user device. In some embodiments, an image reconstruction model may reside at a testing platform. In some embodiments, the user device may transmit data indicating one or more sets of feature vectors to the testing platform. In some embodiments, the testing platform may transmit data representing one or more sets of feature vectors and/or data representative of one or more reconstructed images to a database. In some embodiments, the testing platform may transmit data representative of one or more reconstructed images to a proctor computing device for display on the display of the proctor computing device.

In some embodiments, a feature extraction model may reside at a user device. In some embodiments, an image reconstruction model may reside at a proctor computing device. In some embodiments, the user device may transmit data representing one or more sets of feature vectors to a testing platform. In some embodiments, the testing platform may transmit data representing one or more sets of feature vectors to a database. In some embodiments, the testing platform may transmit data representing one or more sets of feature vectors to the proctor computing device. In some embodiments, the image reconstruction model at the proctor computing device may generate one or more reconstructed images for display on the display of the proctor computing device.

In some embodiments, the feature extraction model may reside on a testing platform. In some embodiments, a user device may transmit data representing one or more images captured by the user device to the testing platform. In some embodiments, transmitting data representing one or more images captured by the user device may not protect user privacy, but may provide other advantages such as, for example, providing data to train feature extraction and/or image reconstruction models. In some embodiments, users may opt in to sharing such information.

In some embodiments, a feature extraction model may reside on the user device. In some embodiments, the user device may transmit to the testing platform data representing sets of extracted feature vectors. In some embodiments, the user device may transmit both data representing sets of extracted feature vectors and data representing images captured by the user device. In some embodiments, transmitting data representing sets of extracted feature vectors from the user device to the testing platform and not transmitting data representing images captured by the user device may be advantageous because, for example, it can reduce the amount of data that is transmitted from the user device to the testing platform. In some embodiments, reducing data usage may reduce the costs associated with taking a test such as, for example, if a user is taking the test on a mobile device that utilizes a metered data plan.

In some embodiments, a testing platform may pass the data received from a user device to a proctor computing device without modification. For example, in some embodiments, the testing platform may receive data representing captured images from the user device and may pass that data to the proctor computing device. In some embodiments, the testing platform may receive data representing sets of feature vectors and may transmit data representing sets of feature vectors to a proctor computing device. In some embodiments, the testing platform may receive data representing reconstructed images from the user device and transmit data representing reconstructed images to the proctor computing device. In some embodiments, passing data through without modification may be advantageous because, for example, doing so may reduce the load on testing platform servers and/or may avoid delays caused by processing.

In some embodiments, an image reconstruction model may be available only on specific computing devices and not on the user device or the proctor computing device. In some embodiments, the image reconstruction model may only be available on a subset of devices of the testing platform. For example, in some embodiments, the image reconstruction model may only be available to computing devices that are used to develop and/or maintain the testing platform. In some embodiments, the image reconstruction model may only be available to computing devices used to train machine learning models, develop additional platform tools, perform analysis, or the like.

In some embodiments, a proctor may only be shown reconstructed images. In some embodiments, showing the proctor only reconstructed images may improve patient privacy as not even the proctor can see the patient. In some embodiments, the user device may only transmit data representing sets of feature vectors to the testing platform. In some embodiments, only sending feature vectors may improve user privacy at least in part because data representing captured images never leaves the user device.

In some embodiments, when reconstructing image frames, additional features may be added. For example, in some embodiments, three-dimensional content may be added. In some embodiments, for example, a message or annotation may be added indicating that a user successfully completed a step that is difficult to track and/or represent (for example, a message or annotation might tell a proctor that the user swabbed their nostrils the correct number of times).

In some embodiments, some portions of the image may be retained. In some embodiments, for example, one or more portions of images that show test kit materials may be included in the reconstructed images that are shown to a proctor and/or may be stored in a database.

In some embodiments, the feature extraction model may be trained to recognize one or more items that should be excluded from the vector space representation such as, for example, medications, other people, framed photos, and other personal items.

In some embodiments, the size of reconstructed images may be dynamically adjusted based on one or more factors such as, for example, network connection, testing platform traffic, user preferences, user input, proctor preferences, proctor input, procedure step, or other factors. For example, more detail may be included for more critical steps in a testing procedure, such as swabbing, adding a reagent, dropping solution onto a test strip, and so forth.

In some embodiments, a physics engine may be used in the image reconstruction process. For example, a physics engine may be used to create reconstructions with smoother motion and less jitter. In some embodiments, the reconstruction model may use skeletal muscular models for more accurate feature mapping. In some embodiments, the image reconstruction model may be able to reconstruct features that are occluded in the images or missing due to processing or network errors (such as, for example, dropped packets). For example, a feature vector can be estimated using a physics engine and/or skeletal muscular model, as both physics and anatomy constrain the placement and/or movement of various features.

As briefly mentioned above, whitening data (e.g., blurring facial features) can be a powerful tool for protecting patient privacy and ensuring regulatory compliance when creating ML models that use patient identifying information (PII). However, such techniques can obscure important information and make it difficult to determine compliance with a testing procedure, detect fraud (e.g., verifying that the same person was present throughout a testing session), and so forth. Accordingly, in some embodiments, a pre-training step can be utilized to replace PII features with derivative indicators (for example, arrows, dots, etc. indicating facial features, normal of facial features, and the like). When such derivative indicators are used, whitened data can maintain salient features for use in ML models, allowing ML models to operate effectively without PII ever being exposed to the model. Such an approach can be applied to training models using protected medical data.

In some embodiments, just in time (JIT) techniques can be used for training a ML model. For example, a small number of test images and/or clips can be buffered to be consumed for ML model training. In some embodiments, the buffered images and/or clips can, in some embodiments, only be accessibly in RAM (e.g., the images and/or clips may not be written to disk or other non-volatile storage). In some embodiments, the location of the images and/or clips in RAM can be static. Thus, adding images or clips to a buffer can overwrite existing images and/or clips. Using such techniques, machine learning training can be performed with minimal data exposure. For example, large amounts of data can be stored in a repository and can be used to train models and curate derivative data without accessing more than a few images or frames of a customer data at a time. Accordingly, data can remain largely secured against some types of data breaches, malicious employees, and so forth. Using conventional methods, large amounts of highly sensitive data can be unencrypted on employee computers, servers, and so forth, which can present significant security and regulatory concerns.

In some embodiments, a system can be configured to use pose data (e.g., six degree of freedom pose data) from a test app on a user device (e.g., an AR-guided test app) in conjunction with object recognition techniques, planar extraction techniques, and so forth to create a 3D model of a customer's test area. In some embodiments, 2D computer vision methods can be used in conjunction with six degree of degree tracking systems to enable scene reconstruction. In some embodiments, the system can use multiview geometry to combine multiple frames from the user's device (e.g., as may be obtained during organic and/or directed user movement) to place and/or track test components such as swabs, vials, test cards, the user's phone, and so forth within a 3D digital reconstruction of the test area.

In some embodiments, the 3D digital reconstruction and tracked objects can be presented to an AI and/or human proctor. The AI and/or human proctor can then use the 3D model and tracked object to provide feedback to the user, for example in the form of arrows, icons, text, and/or other indicates. The feedback can be spatialized in three dimensions and viewable by the user. In some embodiments, the 3D digital reconstruction can be saved and used for machine learning training, customer service calls, training, quality assurance, and so forth. Having access to the 3D digital reconstruction can improve proctor efficiency, ease communication with the user, help to automate test steps, and so forth.

### Asynchronous Testing Efficiency

While the privacy-preservation techniques discussed above can be useful for any type of remotely proctored medical, remote medical visit, or other similar scenario in which PII is exchanged and possibly retained, the privacy-preservation techniques can be especially beneficial for asynchronous remote diagnostic testing, medical visits, and so forth, as discussed in more detail below.

### Semantic Relevance Curation

A doctor visit, especially a follow up visit for treatment can follow a predictable, or often a predetermined script. A doctor may need perform an intake to extract or obtain a plurality of different data points or information from a patient. In some embodiments, the information can include social indicators, biomarkers, side effects, success criteria, or any other information associated with treatment of a particular issue. Various difficulties can arise during a visit that can make it difficult for a doctor or other medical provider to obtain information that is needed or beneficial for treating the patient. Often, time constraints can present significant difficulties.

For example, in some cases, the patient may be rushed through the intake, the patient may be inefficient at communicating the plurality of data points or information, and/or the doctor may not fully understand the plurality of data points or information provided by the patient. Therefore, the doctor may make health care decisions without enough information. In other instances, the patient may provide too much information, which may take up too much of the doctor's time. Additionally, in some cases, the patient may be asked to provide data points or information about rare danger indicators that must be checked every time the patient is provided with treatment from the doctor.

In some embodiments, since the appointment script or questions asked by the doctor may be predictable, or easily determined, the systems, methods, and devices described herein can use artificial intelligence (AI) or machine learning (ML) in order to perform the intake. The AI model can preserve the nuances associated with each patient's medical condition while improving the overall efficiency of the intake process.

In some embodiments, the system can record or capture one or more videos of the patient discussing the plurality of data points or information associated with the patient's medical condition or care. The one or more videos can be captured by a camera or other video capturing device of a patient computing device. In some embodiments, the system can connect the patient to a proctor, a nurse, or other professional via a telehealth conference or session. In some embodiments, the system can connect the patient to one or more prerecorded videos of the doctor. The one or more videos can be, for example, videos of the doctor asking questions associated with the patient's medical condition or care. In some embodiments, a first prerecorded video of the doctor may be displayed to the patient. Based on a response of the patient, the system can automatically and dynamically select a second prerecorded video of the doctor that responds to the response of the patient or asks additional follow up questions to the patient. In some embodiments, the system can use AI to detect the response of the patient. In some embodiments, based on the detected response, the AI can be used to automatically determine the correct second video to display to the patient.

In some embodiments, the system can display a virtual proctor to the patient. The system can automatically and dynamically generate proper responses to the response of the patient. The proper response can be selected from a plurality of premade responses and/or or the system can use a machine learning model in order to automatically and dynamically determine and generate the proper response to the patient.

In some embodiments, the system can decrease an amount of time a doctor must spend with each patient, increasing a number of patients the doctor can see in one day, thereby increasing the efficiency of the doctor's time and/or reducing appointment wait times. Such efficiency improvements can be generally beneficial to doctors and other healthcare providers, but may be especially useful for specialists, where the demand for such specialists can often outstrip the available supply of specialists.

In some embodiments, the system can use a semantic engine. The semantic engine can use machine learning and/or AI to automatically determine salient or important portions of the one or more videos. The system can automatically and dynamically create one or more short self-contained clips. Each clip can contain one or more important or critical pieces of information related to the condition or care of the patient. In some embodiments, the system can automatically send the one or more clips to an available doctor. The doctor can view the one or more clips in a supercut. By sending only the important or critical pieces of information, the available doctor can review and collect the information more efficiently. For example, a patient with a thyroid medication may talk about their symptoms, medication, side effects, or any other related information, and the system may automatically extract or detect several 15 second clips during one or more portions of the video of the patient when the patient discusses energy levels, mental fog, adherence to a medication schedule, etc. The length of the clips can vary. For example, a can be about 5 seconds, about 10 seconds, about 15 seconds, about 30 seconds, about 60 seconds, or any number between these numbers, or more or less depending upon the question, the response, and/or other relevant factors.

In some embodiments, the semantic engine can automatically or conditionally exclude information that is not relevant. For example, during the video recording the system may ask the patient about a rash caused by a medication. If the patient answers that they do not have a rash, the system may exclude that information from the supercut, play a short video or audio clip of the patient saying no rash, or the system may display to the specialist doctor, “no rash.”

In some embodiments, the system or toolchain can include additional features that improve asynchronous patient communication efficiency. In some embodiments, the semantic engine can automatically and dynamically detect and remove pauses in the speech of the patient. In some embodiments, the system can automatically and dynamically adjust a playback speed of the supercut in order to play the patient's speech at a desired words per minute rate. In some embodiments the system can augment the supercut or video clips to display a text log of the interview of the patient. In some embodiments, the doctor can select one or more portions of the text log, and the system can automatically display one or more portions of the video associated with the one or more portions of the text log. In some embodiments, the system can automatically and dynamically detect and group clips that are related. The system can display links to related clips when the doctor is viewing a clip.

In some embodiments, the system can record a response from the doctor after the doctor views the supercut or a portion thereof. In some embodiments, the response can be a prerecorded video with common instructions given by the doctor to patients. In some embodiments, one or more prerecorded responses can be combined with a response recorded after the doctor views the supercut. In this way, the doctor can spend less time responding to the patient.

In some embodiments, the semantic engine can include an input. The input can be a speech-to-text algorithm that can automatically and dynamically create a text log of the one or more video of the patient. The semantic engine can automatically link the test log to a certain timestamp in the one or more videos. In some embodiments, the system can generate one or more semantic hamming distance signals by performing one or more natural language processing tasks. The semantics Hamming distance signal (SHDS) can be a measure of how closely related words, phrases, or sentences are based on the concept or topic of the words or sentences. For example, the word dog can have a low SHDS (e.g., a high semantic closeness) to the word walk, but the word dog can have a high SHDS (e.g., a low semantic closeness) to the word campaign. In some embodiments, the semantic engine can be trained to automatically detect the SHDS using machine learning. In some embodiments, the semantic engine can be trained with and/or analyze medical journals, clinical reports, and/or text logs associated with videos of other patients in order to automatically determine the SHDS of words or sentences. In some embodiments, words or sentences that appear commonly in the same piece of text can have a low SHDS or a high closeness. In some embodiments, words or sentences can have a low SHDS or a high closeness if the words or sentences commonly appear in the same sentence or paragraph.

In some embodiments, the semantic engine can automatically combine the SHDS with other word indicators such as the sentiments of words, keyword matching, natural pauses at the ends of thoughts, etc., in order to generate a signal to represent a correlation or closeness between a first word or sentence to previous words or sentences. In some embodiments, the signal can be a curve. In some embodiments, peaks of the curve can be a focus of a thought, and troughs of the curve can indicate when the patient talks about a new topic or when the patient is done with a thought. In some embodiments, the troughs of the curve can be a focus of a thought, and the peaks of the curve can indicate when the patient talks about a new topic or when the patient is done with a thought.

In some embodiments, the semantic engine can automatically split or divide the text log into one or more discrete thoughts. The system can automatically associate the one or more discrete thoughts with one or more of the questions provided by the system of the doctor. For example, if the doctor or the system asks a question about side effects the system can calculate which thoughts had a semantic closeness to side effects. In some embodiments, thoughts can be included if the thought has a semantic closeness above a closeness threshold. In some embodiments, the system can automatically determine a closeness threshold for each question, or the doctor can input a closeness threshold for each question. In some embodiments, the closeness threshold can be a verbosity limit. For example, the system can calculate that a first thought has a semantic closeness of 97% to side effects, a second thought has a semantic closeness of 80% to side effects, and the rest of the thoughts have a semantic closeness less than 5%. If the closeness threshold is 5%, then only the first thought and the second thought can be included in response to the question.

In some embodiments, the semantic engine can automatically determine the semantic closeness of words or thoughts in real time or substantially real time. In this way, the semantic engine can automatically determine whether the patient has provided enough information to answer a question. If the semantic engine determines that not enough information was provided by the patient, the system can automatically prompt the patient for more information. As described above, there can be many benefits associated with recording patient interactions and operating on said recordings in order to increase efficiency. However, as discussed herein, there can be significant privacy and regulatory risks associated with capturing recordings of a patient. For example, it is important that recordings be stored and/or presenting in a way that does not put PII at risk of exposure by hackers, malicious employees, or even careless employees who may, for example, view recordings of patients in public areas such as cafeterias, coffee shops, and so forth.

Accordingly, the supercuts, individual clips, and so forth discussed herein can comprise reconstructions generated from feature vectors as described herein, thereby enabling efficient review by the provider while preserving patient privacy and reducing the risk that PII is exposed, although some implementations may not use such privacy-preservation techniques.

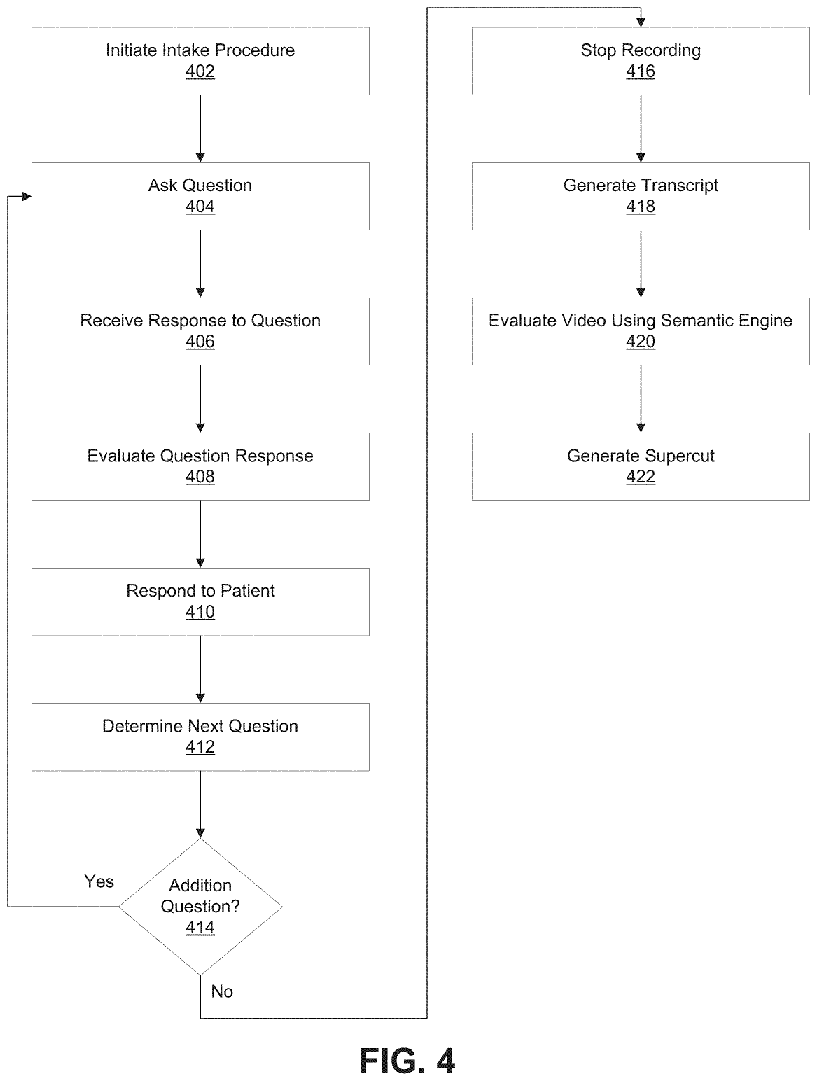

FIG. 4 is a block diagram that illustrates an example embodiment for performing an intake procedure. At block 402, a system can be configured to initiate an intake procedure with a patient. For example, the system can receive a request from the patient to begin a procedure. Initiating the intake procedure can comprise, for example, beginning a recording of the intake procedure. As discussed above, recording the procedure can comprise recording video of the procedure and/or determining and storing feature vectors that can be used to generate a reconstruction of the intake procedure in a manner that preserves patient privacy.

At block 404, the system can ask a first intake question and, at block 406, the system can receive a response from the patient. At block 408, the system can evaluate the patient's response and, based on the patient's response, the system can, at block 410, respond appropriately to the patient. At block 412, the system can determine a next question to ask the patient. For example, if the patient indicated that they are having side effects, the system could inquire about side effects the patient is experiencing, or if the patient indicated they are having trouble complying with the medication schedule, the system could inquire about a number of missed doses, frequency of missed doses, etc. At block 414, the system can determine if there is a next question. If so, the system can return to block 404 and ask the next question. If there are no more questions to ask the patient, the system can advance to block 416 and can stop recording. At block 418, the system can generate a transcript of the intake process. At block 420, the system can use a semantic engine to evaluate the intake. For example, the semantic engine can determine related portions of the intake procedure, identify the most relevant portions of the intake process, and so forth. At block 422, the system can generate a supercut of the intake process that includes that most relevant responses. The supercut can include video (actual video and/or reconstructions), textual, video, and/or audio annotations (e.g., to indicate compliance, the existence or absence of side effects or symptoms, and so forth), and so forth. It will be appreciated that not all steps are necessary. For example, in some implementations, a transcript may be generated and a supercut may not be generated, a supercut may be generated and transcript may not be generating, or neither a transcript nor a supercut may be generated.

Although described primarily in reference to follow up visits, it will be appreciated that the systems and methods described can be used in a wide variety of medical sessions. For example, the systems and methods described above can be used during intake of a new patient, when screening a patient prior to a test or procedure, when performing a remote medical testing session, and so forth.

### Voucher Services

This section describes devices, systems, and methods for voucher service and continuity, such as health testing or diagnostic integrity and continuity. Embodiments of the inventions described herein can comprise several novel features and no single feature is solely responsible for its desirable attributes or is essential to practicing the inventions described.

In some instances, one client or partner, or multiple clients or partners, may be enabled to enabled to purchase a plurality (e.g., a pallet or other plurality of units) of test kit packages. The plurality of test kit packages may be tailored to the client or partner's needs for the test. That is, a generic test kit can be modified to be specifically configured for the client or partner.

In some embodiments, the modified or tailored test kit can include one or more voucher codes. The voucher codes can be generated by a computer system. For example, a system may be configured to generate at least one voucher code (such as a QR code, a bar code, a unique identification number, unique identifier, or the like). The system may further be configured to apply the voucher code to each test kit or plurality of test kits. Upon receipt of at least one test kit package by a user, the user may scan the voucher code to begin preparation of the administration of the test kit. The system may redirect the user to a client or partner specific website to provide the user access to administer the test or tailored test associated with the voucher code.

A client or partner's testing needs may vary depending on the business of the client or partner. Accordingly, client specific, or tailored testing, may be beneficial to provide testing in an efficient manner and/or to administer remote testing and/or collect test results that are relevant to the client or partner's needs. This can more efficiently direct users to administer tests that may be directly related to a client or partner's needs.

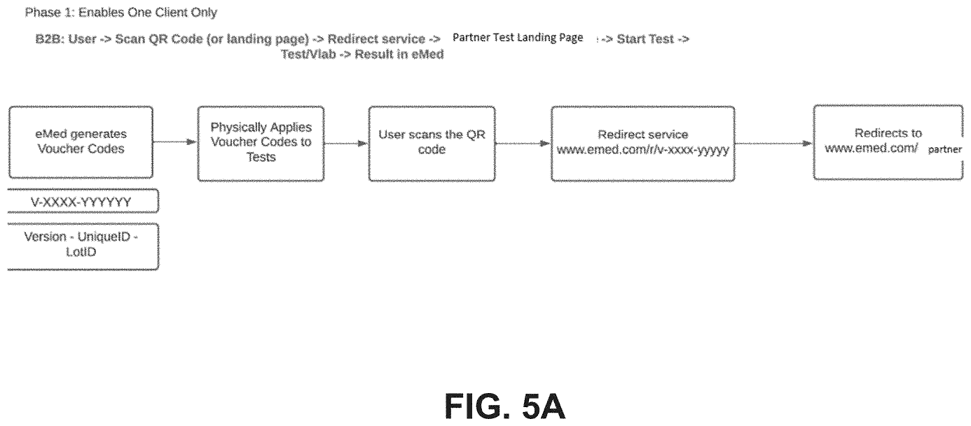

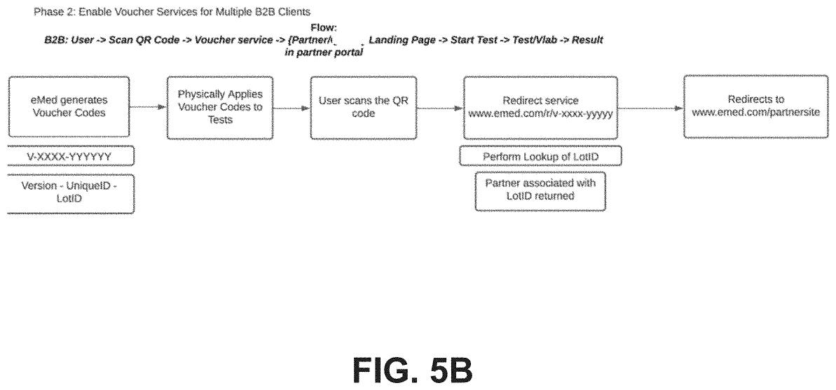

FIG. 5A is a block diagram illustrating an example voucher service protocol or method for a single client who obtains test kits from a testing platform. In this example, the testing platform (e.g., eMed) first generates the voucher codes. In the illustrated example, the voucher codes are in the format “V-XXXX-YYYYY,” where V identifies a test kit version, XXXX identifies a unique code that can be associated with the client, and YYYYY indicates a lot number associated with the test kits. Other forms of the voucher codes are possible, for example, the voucher code can be provided as a QR code or other machine-readable code. The voucher codes can be applied to the test kits, for example, printed or adhered on the packaging of the test kit, provided on an insert within or provided with the test kit, or otherwise. The test kits can then be distributed (e.g., sold) to users.

Next, a user can use the test kit. This can include scanning (or otherwise inputting the voucher code) associated with the test kit. Scanning the voucher code can redirect the user to, for example, a website. The website can be customized based on the specific needs or requirements of the client. For example, in the illustrated embodiment, the user is directed to the website www.emed.com/V-XXXX-YYYYY, where the inclusion of the voucher code directs the user to a customized website (e.g., www.emed.com/parter). In some embodiments, the website is provided by or associated with the testing platform (e.g., eMed). In other embodiments, the website is provided by the client or a third party.

In this way, the client can utilize the services of the testing platform, but provide a tailored experience specific to the client. As an example, the client may be a hotel. The hotel can obtain testing kits from the testing platform that are customized with a hotel specific voucher code. The hotel can distribute the test kit to its guests (or future guests) who can use them to take the tests. When the guests take the tests, they scan the codes and are redirected to a customized experience on the testing platform's website. For example, the customized experience can include hotel branding and information specific to the hotel. At the same time, the customized experience can use the testing platform's service to facilitate administration of the test (e.g., live video proctoring of the test).

FIG. 5B is a block diagram illustrating an example voucher service protocol or method for multiple clients of business-to-business clients. The principles of the illustrated protocols or methods can include the following. In this example, the YYYYY portion of the voucher code is associated with a LotID, and the LotID can be associated with one of a plurality of partners.

Additional detail regarding the use of voucher codes (e.g., with respect to the examples of FIG. 5A, FIG. 5B, or others) is provided below. In some embodiments, a voucher code can be included on test kits as a sticker with a QR code. The voucher code can be applied in other ways and the code can take other forms. In some embodiments, each test kit package may include one, two, or possibly more tests. The QR code may corresponds to a unique URL (e.g., www.emed.com/r/v-xxxx-yyyyyy) where the “v-xxxx-yyyyyy” portion of the URL is what may differ from QR code to QR code. This portion of the URL can be referred to as the “voucher code.” The “v” portion of the voucher code may correspond to a version number, the “xxxx” portion of the voucher code may represent a unique/secret code, and the “yyyyyy” portion of the voucher code may correspond to a lot ID number.

In some embodiments, several test kit packages may be bundled together. All test kit packages contained in a given bundle of test kit packages may be associated with voucher codes having the same lot ID number. Each bundle may also be outfitted with a sticker indicative of lot ID number and/or other information. Several bundles of test kit packages may be assembled onto a single pallet. Each bundle may also be outfitted with a sticker including identification information.

In some instances, when a partner purchases a pallet of test kit packages, a partner identification code may be stored in association with the pallet, all of the bundles of test kit packages in the pallet, and all of the test kit packages in the bundles of test kit packages in the pallet. Each unique URL associated with a test kit package in the pallet may be configured to redirect to another page or site that is associated with the partner. As such, when a user scans a QR code printed on a sticker that has been placed on a test kit package, the user may be taken to the URL corresponding to said QR code and subsequently redirected to another page or site that is associated with the partner.

A user's given testing experience may be tailored to the partner's needs. In addition, this system provides a way for partners to keep track of how many of their tests have been used.

In some embodiments, information beyond just the partner's identity may also be stored in association with test kits. In one example, a cruise line may wish to provide their guests with at-home rapid covid tests that are to be taken prior to boarding a cruise ship. The cruise line can work with a testing platform (such as eMed) and orders several pallets of test kits. The cruise line can ship tests kits to every customer that purchases a cruise ticket. When a cruise line customer decides to use their test kit, they scan the QR code printed on the label on the outside of the test kit package, are directed to the corresponding URL (e.g., www.emed.com/r/v-xxxx-yyyyyy), and subsequently redirected to a special page that has been set up for Carnival (e.g., www.emed.com/carnival). The corresponding URL may also be printed on the sticker in plain text. As such, the user may opt to simply manually enter the URL into their browser instead of scanning the corresponding QR code. The voucher code that is associated with the kit is also parsed and analyzed, and data regarding the usage of the test kit may be relayed to Carnival for tracking purposes, analytics, etc.

In some embodiments, purchase options can include: eMed B2B (B2C, B2B2C), and or OTC vouchered. These can be used to ensure that use of the testing platform (e.g., proctored testing) is paid for. In some instances, vouchers can be purchased separate from inventory (either alone or with generic, unvouchered inventory).

In another example, a process for inbound fulfillment (e.g., eMed B2B) can include one or more of the following steps: eMed receives pallet from manufacturer, which may not be serialized in advance, not in eMed saleable unit, uniform manufacturer lots in receiving boxes; manufacturer boxes are unpacked; individual tests are labeled with voucher codes; the test are assembled into n-count eMed boxes; the n-count boxes are labeled; the labeled n-count boxes are grouped into eMed pallets; the eMed pallets are labeled; and the pallets and/or boxes are stored.

In another example, a process for outbound fulfillment can include one or more of the following steps: receive purchase order (PO); pick and pack order; scan either pallet ID or n-count box ID(s) to associate with PO; print and apply shipping labels; and put on truck to ship.

In some embodiments code-centric requirements and flow can include one or more of the following:
Code may identify the test payor and optionally redirect to their landing page Code may retain value—(e.g., code can be difficult to guess or use without having been given a legit code).
Codes may need a number of usages before expiration based on the minimum quantity per box for that test.
Code may be per-base-unit, not multiple units per pack.
Code, in some embodiments, may not be preassigned to a payor/buyer due to fulfillment before test is repacked into inventory.
   Therefore, code may be able to be assigned to payor/buyer at sell time while associated with test at buy time.
Code can associate to manufacturer lot and expiration date when test kits fulfilled thru testing platform (e.g., eMed).
Code may be able to prove that the test used during sample collection was also the test used during result collection.
Codes can be procedure-specific—(e.g., can't be transferred to another procedure).

### Computer Systems

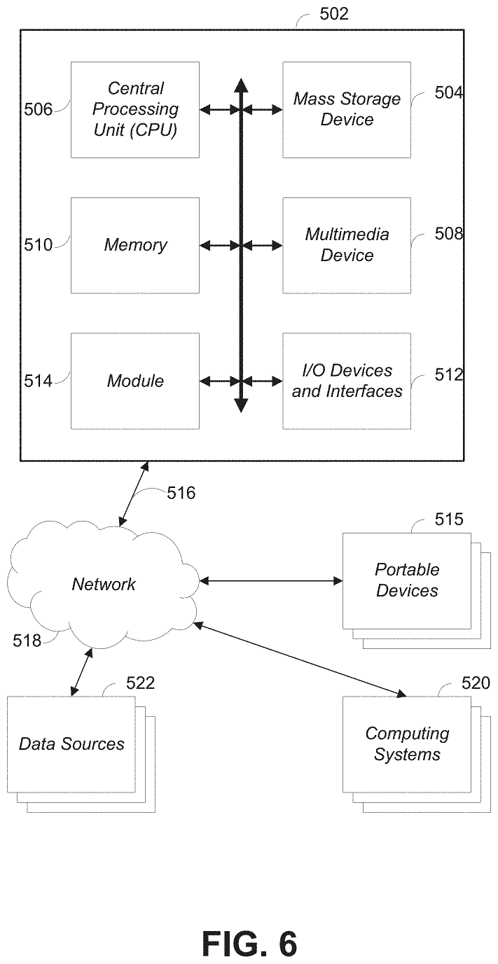

FIG. 6 is a block diagram depicting an embodiment of a computer hardware system configured to run software for implementing one or more embodiments of the health testing and diagnostic systems, methods, and devices disclosed herein.

In some embodiments, the systems, processes, and methods described herein are implemented using a computing system, such as the one illustrated in FIG. 6. The example computer system 502 is in communication with one or more computing systems 520, one or more portable devices 515, and/or one or more data sources 522 via one or more networks 518. While FIG. 6 illustrates an embodiment of a computing system 502, it is recognized that the functionality provided for in the components and modules of computer system 502 may be combined into fewer components and modules, or further separated into additional components and modules.

The computer system 502 can comprise a module 514 that carries out the functions, methods, acts, and/or processes described herein. The module 514 is executed on the computer system 502 by a central processing unit 506 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 502 includes one or more processing units (CPU) 506, which may comprise a microprocessor. The computer system 502 further includes a physical memory 510, such as random-access memory (RAM) for temporary storage of information, a read only memory (ROM) for permanent storage of information, and a mass storage device 504, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 502 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 502 includes one or more input/output (I/O) devices and interfaces 512, such as a keyboard, mouse, touch pad, and printer. The I/O devices and interfaces 512 can include one or more display devices, such as a monitor, that allows the visual presentation of data to a user. More particularly, a display device provides for the presentation of GUIs as application software data, and multi-media presentations, for example. The I/O devices and interfaces 512 can also provide a communications interface to various external devices. The computer system 502 may comprise one or more multi-media devices 508, such as speakers, video cards, graphics accelerators, and microphones, for example.

The computer system 502 may run on a variety of computing devices, such as a server, a Windows server, a Structure Query Language server, a Unix Server, a personal computer, a laptop computer, and so forth. In other embodiments, the computer system 502 may run on a cluster computer system, a mainframe computer system and/or other computing system suitable for controlling and/or communicating with large databases, performing high volume transaction processing, and generating reports from large databases. The computing system 502 is generally controlled and coordinated by an operating system software, such as z/OS, Windows, Linux, UNIX, BSD, SunOS, Solaris, macOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 502 illustrated in FIG. 6 is coupled to a network 518, such as a LAN, WAN, or the Internet via a communication link 516 (wired, wireless, or a combination thereof). Network 518 communicates with various computing devices and/or other electronic devices. Network 518 is communicating with one or more computing systems 520, one or more portable devices 515, and one or more data sources 522. The module 514 may access or may be accessed by computing systems 520, portable devices 515, and/or data sources 522 through a web-enabled user access point. Connections may be a direct physical connection, a virtual connection, and other connection type. The web-enabled user access point may comprise a browser module that uses text, graphics, audio, video, and other media to present data and to allow interaction with data via the network 518.

Access to the module 514 of the computer system 502 by computing systems 520, portable devices 515, and/or by data sources 522 may be through a web-enabled user access point such as the computing systems' 520 or data source's 522 personal computer, cellular phone, smartphone, laptop, tablet computer, e-reader device, audio player, or another device capable of connecting to the network 518. Such a device may have a browser module that is implemented as a module that uses text, graphics, audio, video, and other media to present data and to allow interaction with data via the network 518.

The output module may be implemented as a combination of an all-points addressable display such as a cathode ray tube (CRT), a liquid crystal display (LCD), a plasma display, or other types and/or combinations of displays. The output module may be implemented to communicate with input devices 512 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 502 may comprise a physical or logical connection established between a remote microprocessor and a mainframe host computer for the express purpose of uploading, downloading, or viewing interactive data and databases online in real time. The remote microprocessor may be operated by an entity operating the computer system 502, including the client server systems or the main server system, an/or may be operated by one or more of the data sources 522, one or more of the computing systems 520, and/or one or more of the portable devices 515. In some embodiments, terminal emulation software may be used on the microprocessor for participating in the micro-mainframe link.

In some embodiments, computing systems 520 or portable devices 515 who are internal to an entity operating the computer system 502 may access the module 514 internally as an application or process run by the CPU 506.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 502 may include one or more internal and/or external data sources (for example, data sources 522). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 502 may also access one or more databases 522. The databases 522 may be stored in a database or data repository. The computer system 502 may access the one or more databases 522 through a network 518 or may directly access the database or data repository through I/O devices and interfaces 512. The data repository storing the one or more databases 522 may reside within the computer system 502.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein, but are to be accorded the widest scope consistent with this disclosure, the principles and the novel features disclosed herein.

What is claimed is:

1. A method comprising:
receiving, by a computing system, from a user device, a request to begin a remote medical session;
initiating, by the computing system, the remote medical session;
receiving, by the computing system, from the user device, a plurality of feature vectors representative of one or more images; and
generating, by the computing system, one or more reconstructed images using the received feature vectors.

2. The method of claim 1, further comprising:
transmitting, by the computing to a proctor computing device, the one or more reconstructed images.

3. The method of claim 1, further comprising:
storing, by the computing system in a non-volatile memory, at least one of the plurality of feature vectors of the one or more reconstructed images.

4. The method of claim 1, further comprising:
generating, by the computing system based at least in part on the one or more reconstructing images, a video.

5. The method of claim 4, wherein generating the video comprises applying a physics engine to the plurality of feature vectors.

6. The method of claim 4, wherein generating the video comprises applying a skeletal muscular model to the plurality of feature vectors.

7. The method of claim 4, wherein generating the video comprises estimating one or more missing feature vectors.

8. The method of claim 7, wherein the estimating is performed using at least one of a physics engine or a skeletal muscular model.

9. The method of claim 1, wherein generating the one or more reconstructed images comprises:
detecting one or more objects to exclude from the one or more reconstructed images; and
excluding the one or more objects from the reconstructed images.

10. The method of claim 1, further comprising:
receiving, by the computing system from the user device, audio of the remote medical session.

11. The method of claim 10, further comprising:
generating, from the received audio, a transcript.

12. The method of claim 10, wherein the audio comprises one or more user responses to one or more questions, further comprising:
determining, by the computing system using a semantic engine, a beginning of a user response;
determining, by the computing system using the semantic engine, an end of the user response.

13. The method of claim 12, further comprising:
determining, by the computing system using the semantic engine, a type of the user response.

14. The method of claim 1, further comprising:
providing, by the computing system to the user, a question;
receiving, by the computing system from the user, a response to the question;
evaluating, by the computing system, the received response; and
providing, by the computing system based at least in part on the evaluation, a response to the user.

15. The method of claim 14, further comprising:
determining, by the computing system based at least in part on the user response, a second question; and
providing, by the computing system to the user, the second question.

16. The method of claim 15, further comprising:
determining, by the computing system, that there are no more questions to ask the user.

17. The method of claim 16, further comprising:
generating, by the computing system, a transcript of the user responses.

18. The method of claim 16, further comprising:
evaluating the user responses using a semantic engine.

19. The method of claim 16, further comprising:
generating, by the computing system, a supercut comprising at least part of one or more user responses.

20. The method of claim 1, further comprising:
receiving, by the computing system from the user device, one or more image frames; and
training a machine learning model to extract feature vectors from the one or more image frames.
